(19) United States
(12) Patent Application Publication
(10) Pub. No.: US 2008/0275229 A1
(43) Pub. Date: Nov. 6, 2008

(54) Method for increasing the speed of nucleic acid amplification reactions

(75) Inventors: Paul Lem, Ottawa (CA); Sebastien Fournier, Ottawa (CA); Caitlin Ritz, Ottawa (CA); Chris Harder, Gatineau (CA); Alan Shayanpour, Stittsville (CA)

Correspondence Address: PYLE & PIONTEK;ATTN: THOMAS R. VIGIL, 221 N LASALLE STREET , ROOM 2036, ROOM 2036, CHICAGO, IL, 60601, US

(73) Assignee: Spartan Bioscience, Inc., Ottawa (CA)

(21) Appl. No.: 11/975,439

(22) Filed: Oct. 19, 2007

(30) Foreign Application Priority Data
May 2, 2007 (CA) 2,587,198

(51) Int. Cl. C07H 21/04

(52) U.S. Cl. 536/25.42

(57) ABSTRACT

The speed of a nucleic acid amplification reaction, such as the Polymerase Chain Reaction (PCR), can be increased by setting the temperature of heat sources above and below the desired denaturation, annealing, and extension temperatures. The reaction mixture is only contacted with the heat sources long enough for the desired temperatures to be reached.

### BACKGROUND OF THE INVENTION

1. Field of the Invention

This invention relates to methods for increasing the speed of nucleic acid amplification reactions.

2. Description of the Prior Art

It is believed that nucleic acid amplification reactions, e.g., the Polymerase Chain Reaction (PCR), require a uniform thermal gradient in order to be successful. For example, Neumaier et al (see Neumaier, M., Braun, A., and Wagener, C. (1998); “Fundamentals of Quality Assessment of Molecular Amplification Methods in Clinical Diagnostics”, Clinical Chemistry. 44(1): 12-26.) teach that “Uniform temperature transition is an important aspect for a successful amplification” and “The homogeneity of heat conduction in the reaction block is of crucial importance. The heat performance of the cycler and the uniformity of heat conduction in the heating block must be controlled regularly to avoid false negative results.”

Consequently, manufacturers of PCR machines have engineered their instruments to generate uniform thermal gradients. For example, in 1992, Stratagene introduced the RoboCycler™ temperature cycler, a unique four-block instrument that claimed to achieve unparalleled temperature uniformity (see Renzi, P., Danssaert, J., Hayfield, J., Rahilly, M., Jerpseth, B. (1992); Strategies.)5(2): 41-42.

More recently, Corbett Research developed the Rotor-Gene™ instrument that heats and cools PCR reaction tubes via air jets. The rotor containing the reaction tubes spins at very high speeds, and the stated intention is to increase temperature uniformity.

Additionally, there are products, e.g., the DRIFTCON™ system (Appropriate Technical Resources, Inc.), which enables researchers to test the temperature uniformity of the thermal block in their PCR machines.

In a Polymerase Chain Reaction (PCR), the maximum denaturation temperature is recommended not to exceed 95-96° C. (see Gelfand, D. H. and White, T. J. 1990. “Thermostable DNA Polymerases.” In PCR Protocols: A Guide to Methods and Applications.” Innis, M. A., Gelfand, D. H., Sninsky, J. J., and White, T.J., eds. San Diego: Academic Press. 129-141.) The reason suggested was because higher denaturation temperatures was said to lead to heat inactivation of the polymerase and failure of the PCR (see Hendrikx, Tom, Verblakt, Marc, Pierik, Roger. (2001). “The Impact of the Temperature Performance of Thermal (PCR) Cyclers on the Generated Results, and the Obligation For Regular Validation of PCR Thermal Cyclers.” CYCLERtest BV, Landgraaf, The Netherlands.”). Not surprisingly, then, 99.9° C. is the maximum denaturation temperature which can be programmed into the GeneAmp®9700 PCR System from Applied Biosystems. The GeneAmp® machine is an example of a Peltier-controlled thermal cycling device in which the temperature of a metal block is ramped up and down in temperature. Reaction tubes may be inserted into the metal block, and the contents of the reaction vessel ramp up and down in temperature with the block. The ramp time of the GeneAmp® 9700 machine ranges from about 3 to about 5° C./second.

For successful PCR, it is not necessary to hold the reaction mixture for longer than 1 second at the denaturation or annealing temperatures. For example, Wittwer and Garling demonstrated that optimal denaturation at 92°-94° C. occurred in less than one second, and annealing for one second or less at 54°-56° C. gave the best product specificity and yield. (see Wittwer C T, Garling D J. (1991). “Rapid Cycle DNA Amplification: Time and Temperature Optimization.” Biotechniques. 10(1):76-83.

Traditionally, PCR has been performed with cycles of three temperatures. For example, a temperature of 95° C. for denaturation, a temperature of 56° C. for annealing, and a temperature of 72° C. for extension. It is known that the annealing and Haedicke et al. developed a PCR assay for Salmonella that used two-temperature PCR. (see Haedicke W et al. (1996). “Specific and Sensitive Two-Step Polymerase Chain Reaction Assay For the Detection of Salmonella species.” Eur J Clin Microbiol Infect Dis. 15(7):603-607.).

They used a denaturation temperature of 94° C. for 30 seconds and a combined annealing/extension step at 70° C. for 1 minute. The thermal cycler which was used was a TC 9600 system (Perkin-Elmer Cetus). This apparatus used Peltier-controlled heating and cooling to ramp a metal block up and down in temperature. Specifically, a reaction vessel would have been placed in the metal block, and then the temperature of the block would have been ramped up to 94° C. and then held at that temperature for 30 seconds. Heat would have been transferred from the metal block to the reaction mixture inside the reaction vessel.

The invention in its general form will first be described, and then its implementation in terms of specific embodiments will be detailed with reference to the drawings following hereafter. These embodiments are intended to demonstrate the principles of the invention, and the manner of its implementation. The invention in its broadest sense and more specific forms will then be further described, and defined, in each of the individual claims which conclude this specification.

### SUMMARY OF THE INVENTION

### Statement of Invention

A broad aspect of the present invention provides a method of performing a nucleic acid amplification reaction where a reaction mixture is subjected sequentially to a selected denaturation temperature which is provided by a heat source and to a selected annealing, and/or extension temperature which includes the step of moving the reaction mixture out of the influence of the heat source once the temperature is higher than the desired denaturation temperature and is lower than the desired annealing, and/or extension temperature.

### Other Features of the Invention

By one variant thereof, the method includes establishing a non-uniform temperature gradient across the reaction mixture.

By a second variant thereof, the method includes the steps of first setting the temperature of the heat source higher than the desired denaturation temperature, and setting the temperature of the heat source lower than the desired annealing, and/or extension temperatures, bringing the temperature of the reaction mixture to the desired temperature through the influence of the heat source(s), and moving the reaction mixture is moved out of the influence of the heat source once the desired temperature is reached.

By a variation thereof, the reaction mixture is brought to the desired temperature by direct contact with the heat source.

By another variant thereof, the method includes setting the temperature of a first heat source to about 15° C. higher than the desired denaturation temperature, and subjecting the reactions mixture to the influence of that temperature, whereby the temperature of the reaction mixture is brought up to about 95° C. in about 30 seconds.

By a variation thereof, the reaction mixture is caused to increase in temperature by 23° C. (65° C. to 88° C.) in only 13 seconds, i.e., at about 1.8° C./second.

By another variant thereof, the method includes setting the temperature of a second heat source to about 10° C. lower than the desired annealing, and/or extension temperatures, and subjecting the reactions mixture to the influence of that temperature for about 13 seconds, whereby the temperature of the reaction mixture is brought down to about 65° C. in about 18 seconds.

The foregoing summarizes the principal features of the invention and some of its optional aspects. The invention may be further understood by the description of the preferred embodiments, in conjunction with the drawings, which now follow.

### BRIEF DESCRIPTION OF THE DRAWINGS

In the accompanying drawings.

FIG. 1 is a top plan view of a reaction vessel holder for carrying out a method according to one embodiment of the present invention;

FIG. 2 is an isometric view of the reaction vessel holder shown in FIG. 1;

FIG. 3 is an isometric diagram of a mathematical model when carrying out a method according to one embodiment of the present invention;

FIG. 4 is a diagram of finite analysis results, showing the fluid temperature in a reaction tube after 20 seconds of partial contact with a 95° C. heater block when carrying out a method according to one embodiment of the present invention; and

FIG. 5 is a diagram of finite analysis results, showing the fluid temperature in a reaction tube after 20 seconds of full contact with a 95° C. heater block when carrying out a method according to one embodiment of the present invention.

### DESCRIPTION OF PREFERRED EMBODIMENTS

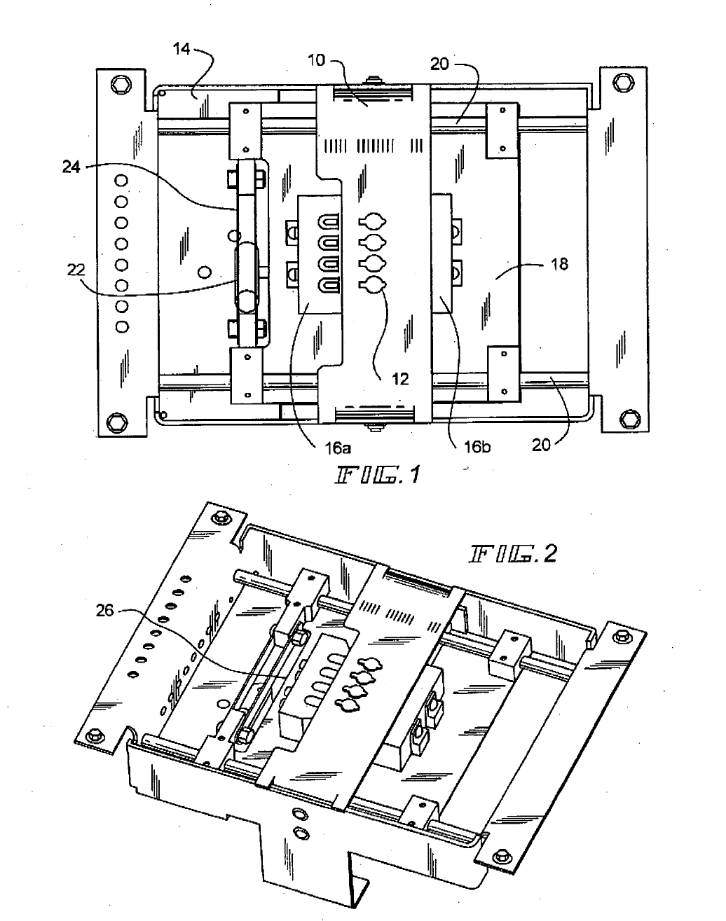

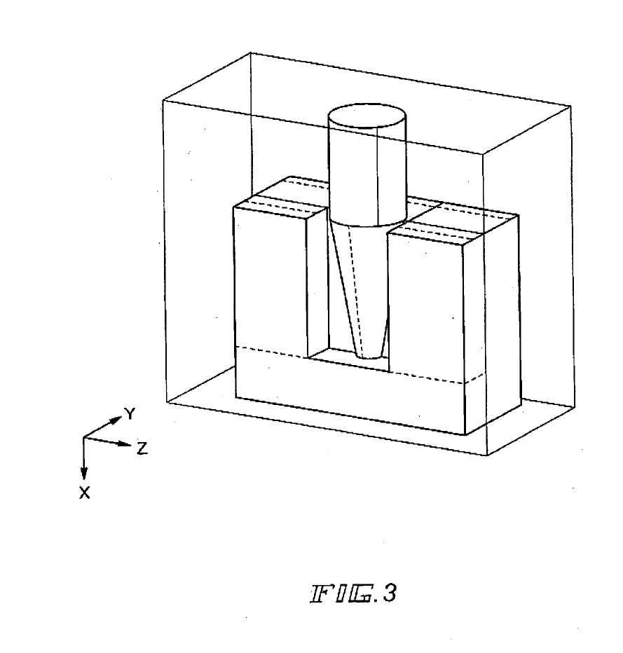

A thermal cycling device with two fixed temperature heat blocks was constructed based on the principles described in U.S. Patent Application 60/563,061, but modified as follows as shown in FIG. 1 and FIG. 2.

A holder 10 for the reaction vessels 12 was constructed by drilling holes of the appropriate size into a chassis 14 comprising a flat sheet of metal. The reaction vessel holder 10 was bolted onto chassis 14. Underneath the reaction vessel holder 10, proximal heater block 16a, and distal heater block 16b were affixed to a support board 18. Grooves (not seen) were machined into the proximal heater block 16a, and distal heater block 16b, the grooves being shaped precisely to fit the shape of the reaction vessels 12. The proximal heater block 16a, and distal heater block 16b also contained resistive heaters (not seen) which were controllable to maintain a set temperature. The support board 18 was configured and arranged to be able to move in one dimension by sliding along two metal shafts 20. Motion of the support board 18 was driven by a cam shaft 22 which was configured and structured to be rotatable in one direction by a motor (not seen). The cam shaft 22 was thus configured to rotate in between a pair of plastic or metal leaf springs 24. The cam shaft 22 was configured and structured to have three main positions, namely: (1) pointing parallel away from the proximal heater block 16a, and distal heater block 16b to result in a configuration where the distal heater block 16b came into contact with the reaction vessels 12; (2) pointing perpendicular to the proximal heater block 16a, and distal heater block 16b to result in a configuration where neither of the proximal heater block 16a, and distal heater block 16b were in contact with the reaction vessels 12; and (3) pointing parallel towards the proximal heater block 16a, and distal heater block 16b to result in a configuration where the proximal heater block 16a came into contact with the reaction vessels 12.

Thus, when the cam shaft 22 were in the 2^{nd} position, the reaction vessels 12 are not in contact with either the proximal heater block 16a, nor the distal heater block 16b. If the middle section 26 were to be cut out of the support board 18, then this middle position 26 would be convenient for imaging the bottom of the reaction vessels 12. Specifically, a blue LED light source may be shone at the bottom of the reaction vessel to excite the contents of the vessel, e.g. SYBR® Green Dye (Molecular Probes, Inc.). Emitted light from the vessel may be detected by means of a CCD camera. To filter out blue light from the LED source, a bandpass filter may be placed in front of the CCD camera so that only higher wavelengths e.g., green and red are allowed to pass through. This helps improve the signal-to-noise ratio.

The use of a cam shaft 22 with the leaf springs 24 attached to the support board 18 helps ensure good contact between the proximal heater block 16a, and distal heater block 16b and the reaction vessels 12. The reason is because the cam shaft 22 is able to deflect the leaf springs 24 when the cam shaft 22 is parallel to, and facing either towards or away from the proximal heater block 16a, and distal heater block 16b. This enables the cam shaft 22 to exert extra force, thereby to drive the proximal heater block 16a, and distal heater block 16b into contact with the reaction vessels 12, and to correct for dimensional tolerances.

It is important to note that the according to certain aspects of the present invention the reaction vessels 12 only come into partial contact with the proximal heater block 16a, and distal heater block 16b. This means that there is a non-uniform (i.e. non-zero) temperature gradient across the reaction vessel 12. The reason is because, although the proximal heater block 16a, and distal heater block 16b are set at a certain temperature, the top of the reaction vessels 12 experience a different temperature because it is held in place by a material which serves as a passive insulator, and the side walls of the reaction vessels 12 which are not in contact with the proximal heater block 16a, and distal heater block 16b are exposed to the temperature of the ambient air.

Using this apparatus according to an apparatus aspect of the present invention, it is possible to perform a two-temperature nucleic acid amplification reaction, e.g., the Polymerase Chain Reaction (PCR). For example, the proximal heater block 16a may be set at 95° C. to enable denaturation of the DNA template in the reaction mixture. The distal heater block 16b may be set at 60° C. to enable the combined step of primer annealing and extension.

In one general procedure according to one method aspect of the present invention, thermocouples were inserted into a reaction vessel 12 containing an aqueous reaction mixture and the temperature was monitored. It was determined that it took at least 30 seconds for the aqueous reaction mixture in the centre of the reaction vessel 12 to reach the permissive denaturing temperature once the proximal heater block 16a which was set at 95° C. was moved into contact with the reaction vessel 12. Similarly, it was determined that it took at least 30 seconds for the temperature in the reaction vessel 12 to go down to a permissive annealing temperature once the distal heater block 16b which was set at 60° C. was moved into contact with the reaction vessel.

Alternatively, in another general procedure according to one method aspect of the present invention, it was discovered that it was possible to perform a successful two-temperature PCR by setting the proximal heater block 16a, and distal heater block 16b at temperatures other than the traditional 94-96° C. denaturation temperature. Specifically, it was discovered that it was possible to set the proximal heater block 16a at 110° C., which was about 15° C. higher than the desired denaturation temperature. Then, it was discovered that it was possible to bring the reaction mixture up to the permissive denaturing temperature by contacting the reaction vessel 12 containing the reaction mixture with the proximal heater block 16a for only 13 seconds. In other words, the reaction vessel 12 was contacted with the proximal heater block 16a for a duration which was sufficient for the reaction mixture to reach a temperature permissive for productive PCR, but not long enough for it to equilibrate and to reach the 110° C. temperature of the proximal heater block 16a. In this case, the temperature of the reaction vessel 12 reached 88° C. in only 13 seconds, less than half the time required when compared to setting temperature of the proximal heater block 16a at 95° C. This represents a large savings in temperature ramp time. Specifically, the reaction mixture increases in temperature by 23° C. (65° C. to 88° C.) in only 13 seconds i.e. 1.8° C./second.

Similarly, it was discovered that it was possible to set the temperature of the distal heater block 16b at 50° C. This enabled the temperature of the reaction mixture in the reaction vessel to come down to 65° C. after only 18 seconds of contact with the distal 50° C. heater block 16b which was set at 50° C.

### EXAMPLES

The following experiment is intended to demonstrate the theoretical basis for the method of aspects of the present invention

### Experiment 1

### Finite Element Analysis of Temperature Gradient

In embodiments of the method of the present invention, the reaction vessels come into partial contact with the proximal and distal heater blocks. In other words, one side of a reaction vessel is in contact with a heater block and one side of that reaction vessel is exposed to ambient air conditions. As well, the top of a reaction vessel is exposed to ambient air conditions. Therefore, one would expect there to be a temperature gradient from top to bottom and from side to side of the reaction vessels.

To test this hypothesis, Finite Element Analysis (FEA) was performed using Icepak® (Fluent Inc.), a software package for Computational Fluid Dynamics (CFD).

With the FEA model, the temperature gradient of the reaction liquid inside a reaction tube was modeled for two conditions, namely: (1) the reaction vessel in partial contact with 95° C. a heater which was set at 95° C.; and (2) the reaction vessel in full partial contact with a heater which was set at 95° C.

The following assumptions were used in the model:

| Material | Density | Specific Heat | Thermal Conductivity |
|---|---|---|---|
| Properties | [kg/m3] | [J/kg k] | [W/mk] |
| Ambient Air: | 1 | 1008 | 0.028 |
| Water | 967 | 4260 | 0.67 |
| Plastic | 1200 | 1527 | 0.24 |
| Copper | 8960 | 385 | 385 |

| Cuvette Geometry | Measurement (mm) |
|---|---|
| Total Height | 20.0 |
| Cone Height | 12.0 |
| Cone Top (outside diameter) | 6.0 |
| Cone Bottom (outside diameter) | 2.0 |
| Material Thickness | 0.3 |

| Computational | |
|---|---|
| Domain Details | Measurement (mm) |
| Height | 30.0 |
| Width | 30.0 |
| Depth | 15.0 |
| Ambient Temp. | 50.0 |

Additional assumptions included the following:

Four side walls of the domain are open to ambient. This simulates the fact that the air is allowed to move on the side surfaces of the heater.

Bottom side of the domain is adiabatic, which simulates the poor conductivity of the electronic board used to support the heaters.

Top side of the domain is adiabatic, which simulates that the insulated cover will be placed on top of the four cuvettes.

Heat sources are mounted to the back of the copper heaters with the assumption of no thermal resistance between the heat source and the copper block.

Heat source is maintained at 95° C.

Liquid volume is 30 μL. A solid block with the properties of air sits on top of the fluid. This technique is used to avoid the free surface condition that most CFD packages can't handle.

A diagram of this mathematical model is depicted in FIG. 3.

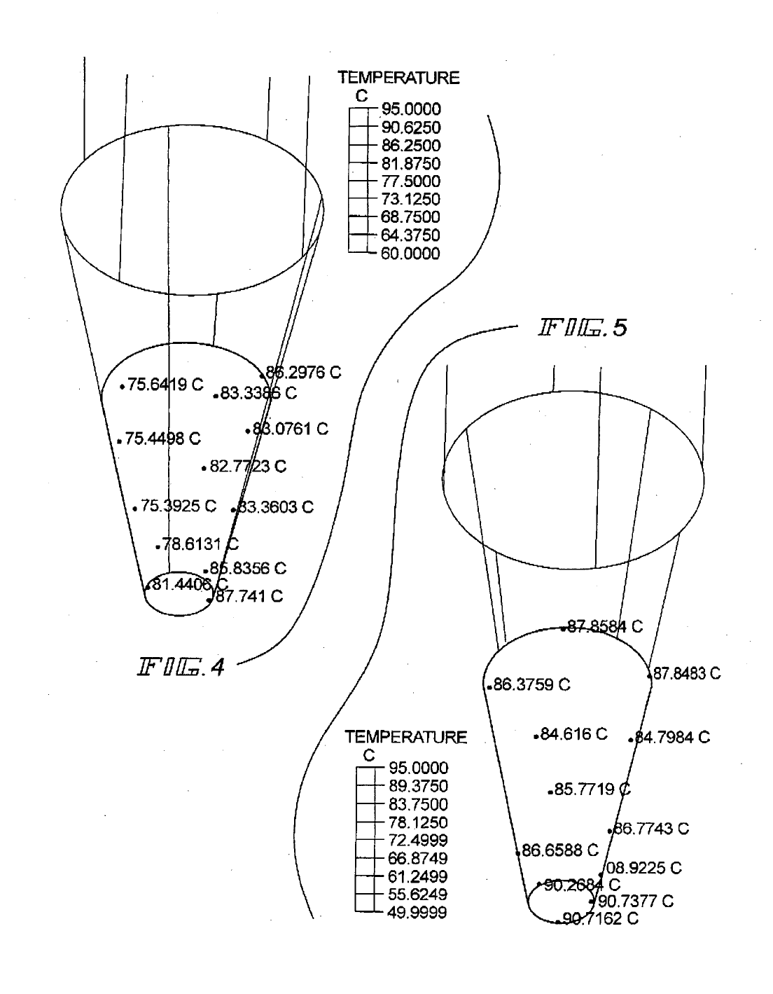

On conducting the above experiment it was found that when the reaction tube was in partial contact with the heater, (as shown diagrammatically in FIG. 4) there was a large temperature gradient from side to side, and from top to bottom, in the liquid in the reaction vessel. From side to side, the gradient ranged from about 75-83° C. From top to bottom, the gradient ranges from about 75-87° C.

In contrast, when the reaction vessel was in full contact with the heater, (as shown diagrammatically in FIG. 5) there was only a temperature gradient from top to bottom in the liquid in the reaction tube. From top to bottom, the gradient ranges from about 84-91° C.

The following are examples of the method of aspects of the present invention

### Example 2

### Temperature Measurement of Fluid in Reaction Tube

The method in this example was carried out using thin-walled 0.2 mL polypropylene tubes (Axygen Scientific). These tubes were filled with 20 μL of distilled water, and topped with 15 μl of mineral oil. Holes of about 1-mm in diameter were drilled into the tops of the tubes, and a pipette tip was glued to the hole and positioned such that at its narrowest end, the tip sat in the centre of the tube, 2 mm from the bottom and equidistant from the sides. A temperature-sensing thermocouple (Omega, Part#5SRTC-TT-T-40-72) was threaded through the hole in the tube lid, and pipette tip to be positioned in the centre of the volume of liquid within the tubes. Temperature data from the thermocouple was logged with a Data Logger (Fluke, Hydra Data Logger, Model 2625).

The reaction tube containing the thermocouple was moved into partial contact with a 110° C. heater for 15 seconds using the apparatus of one aspect of the present invention as described above. Then, the tube was moved into partial contact with a heater which was set at 50° C. for 5 seconds. This process of alternating partial contact with a heater which was set at 110° C. and a heater which was set at 50° C. was repeated several times to allow the high and low temperatures reached by the liquid to reach equilibrium values.

Next, the reaction tube containing the thermocouple was moved into partial contact with a heater which was set at 100° C. for 20 seconds using the apparatus of one aspect of the present invention as described above. Then, the tube was moved into partial contact with a heater which was set at 60° C. for 10 seconds. This process of alternating partial contact with the heater which was set at 100° C. and the heater which was set at 60° C. was repeated several times to allow the high and low temperatures reached by the liquid to reach equilibrium values.

It was found that when the tube was in partial contact with the heater which was set at 110° C. for 15 seconds, the liquid reached a maximum temperature of 87.1° C. It was found that when the tube was in partial contact with the heater which was set at 50° C. for 5 seconds, the liquid reached a maximum temperature of 71° C. Note that the measured temperatures represent the average temperature of the fluid in the tube. Based on the thermal model described in Example 1, there would actually be a thermal gradient across the fluid in the tube.

It was found that when the tube was in partial contact with the heater which was set at 100° C. for 20 seconds, the liquid reached a maximum temperature of 82.8° C. It was found that when the tube was in partial contact with the heater which was set at 60° C. for 10 seconds, the liquid reached a maximum temperature of 66.7° C.

One prior art way of heating up a reaction mixture to a desired denaturation temperature is to contact it with a heater block at a fixed temperature and hold it there long enough for the mixture to reach thermal equilibrium with the heater block. However, as demonstrated above, by carrying out a method according to an aspect of the present invention, as demonstrated by the above example a significant improvement results.

In practicing a method according to an aspect of the present invention, the heater block was set at a temperature higher than the desired denaturation temperature, and the reaction mixture was moved out of contact with the heater block once the desired temperature was reached, but before the mixture reached thermal equilibrium with the heater block. Specifically, the desired denaturation temperature was in the range from 90-95° C., and the temperature of the heater block was set higher than this temperature at 100° C. or 110° C. Then, the reaction tube was contacted with the heater block only for the duration required for the reaction mixture to reach 90-95° C., and not long enough for the mixture to equilibrate at 100° C. or 110° C. with the heater block.

### Example 3

### Two-Temperature PCR With High and Low Temperatures

Using the thermal cycling apparatus according to an apparatus aspect of the present invention, the Polymerase Chain Reaction (PCR) was performed in a 20 μl volume with 1.5 μg template DNA prepared by boiling lysis from a colony of Methicillin-resistant Staphylococcus aureus (MRSA). The reaction mixture also contained 0.125 mM of each deoxynucleotide 1 μM of each oligonucleotide primer, 50 mM KCl, 10 mM Tris-HCl (pH 8.5), and 2.5 mM magnesium chloride. The primers were designed against the dnaC helicase gene (forward primer sequence 5′-cgagatttagaccaaatgacag-3′ and reverse primer sequence of 5′-atcatacgtgtggctaactgat-3′). The expected amplicon size was 188 base pairs. Thermus aquaticus polymerase (1 Unit of Taq polymerase, Invitrogen) was added, the samples placed in a typical 0.2 ml thin-walled PCR tube (Axygen Scientific, Inc.).

### Example 3

### Experiment 1

In the first experiment, one of the heater blocks was set at 110° C. and the reaction mixture underwent an initial denaturation step where the PCR tubes were contacted with the heater block which was set at 110° C. for 5 seconds. Then, the mixture was cycled 35 times through denaturation (110° C. temperature of first heater block) and annealing/extension (55° C. temperature of second heater block). During each cycle, the reaction tube was contacted with the heater block which was set at 110° C. for 5 seconds and with the heater block which was set at 55° C. for 5 seconds. Amplification products were fractionated by electrophoresis on a 1.5% agarose gel. Total run time for initial denaturation and 35 cycles of PCR was about 13.5 minutes.

### Example 3

### Experiment 2

In the second experiment, the same reaction mixture was placed in the thermal cycling device and underwent an initial denaturation step where the PCR tube was contacted with the heater blocks was set at 95° C. for 45 seconds. Then, the mixture was cycled 35 times through denaturation (95° C. temperature of first heater block) and annealing/extension (55° C. temperature of second heater block). During each cycle, the reaction tube was contacted with the heater block which was set at 95° C. for 5 seconds and with the heater block which was set at 55° C. for 5 seconds. Amplification products were fractionated by electrophoresis on a 1.5% agarose gel. Total run time for initial denaturation and 35 cycles of PCR was about 14 minutes.

### Example 3

### Experiment 3

In the third experiment, the same reaction mixture was placed in the thermal cycling device and underwent an initial denaturation step where the PCR tube was contacted with the heater block which was set at 95° C. for 30 seconds. Then, the mixture was cycled 35 times through denaturation (95° C. temperature of first heater block) and annealing/extension (55° C. temperature of second heater block). During each cycle, the reaction tube was contacted with the heater block which was set at 95° C. for 20 seconds and with the heater block which was set at 55° C. for 20 seconds. Amplification products were fractionated by electrophoresis on a 1.5% agarose gel. Total run time for the initial denaturation and 35 cycles of PCR was about 25 minutes.

In experiment 1, electrophoresis showed that the expected 188-base-pair amplicon was produced. In experiment 2, no amplification product was observed with electrophoresis. In experiment 3, the expected 188-base-pair amplicon was observed with electrophoresis. In all experiments, about 10 μl of the reaction mixture evaporated onto the sides and top of the reaction tube, leaving about 10 μl remaining in the bottom.

The results showed that setting the temperature of the heater block at 110° C. (15° C. above the desired temperature of 95° C.) enabled the reaction tube to achieve an effective annealing temperature after being held in contact with the heater block for only 5 seconds.

In contrast, it was found that it was not effective to set the temperature of the heater block at 95° C. and to contact it with the reaction tube for 5 seconds. Instead, a successful reaction only occurred when the tube was held in contact with the heater block heater block which was set at 95° C. for 20 seconds.

These results indicate that temperature overshoots decrease the run-time of PCR amplification.

### Example 4

### Two-Temperature PCR With High and Low Temperatures on RoboCycler®

Polymerase Chain Reaction (PCR) was performed with the same reaction mixture as described in Example 2 using a RoboCycler® 96 temperature cycler without a heated lid (Stratagene). Each of the reactions was overlaid with 10 μL of mineral oil to prevent evaporation.

The RoboCycler® consists of a robotic arm which moves reaction tubes into contact with fixed temperature heater blocks. Unlike the thermal cycling apparatus according to an apparatus aspect of the present invention, the heater blocks of the RoboCycler® contain “wells” for the reaction tubes which surround them on all sides. Only the top of the reaction tube is not in contact with the heater blocks. With the RoboCycler®, the maximum temperature which can be set for the hottest block is 99° C.

### Example 4

### Experiment 1

In experiment 1, one of the heater blocks was set at 99° C. and the reaction mixture underwent an initial denaturation step where the PCR tubes were contacted with the heater block which was set at 99° C. for 10 seconds. Then, the mixture was cycled 35 times through denaturation (99° C. temperature of first heater block) and annealing/extension (55° C. temperature of second heater block). During each cycle, the reaction tube was contacted with the heater block which was set at 99° C. for 5 seconds and with the 55° C. heater block for 5 seconds. Amplification products were fractionated by electrophoresis on a 1.5% agarose gel. Total run time for initial denaturation and 35 cycles of PCR was 9.5 minutes.

### Example 4

### Experiment 2

In experiment 2, the same reaction mixture was placed in the thermal cycling device and underwent an initial denaturation step where the PCR tube was contacted with the heater block which was set at 95° C. for 10 seconds. Then, the mixture was cycled 35 times through denaturation (95° C. temperature of first heater block) and annealing/extension (55° C. temperature of second heater block). During each cycle, the reaction tube was contacted with the heater block which was set at 95° C. for 5 seconds and with the heater block 55° C. for 5 seconds. Amplification products were fractionated by electrophoresis on a 1.5% agarose gel. Total run time for initial denaturation and 35 cycles of PCR was 9.5 minutes.

### Example 4

### Experiment 3

In experiment 3, the same reaction mixture was placed in the thermal cycling device and underwent an initial denaturation step where the PCR tube was contacted with the heater block which was set at 99° C. for 10 seconds. Then, the mixture was cycled 35 times through denaturation (99° C. temperature of first heater block) and annealing/extension (55° C. temperature of second heater block). During each cycle, the reaction tube was contacted with the heater block which was set at 99° C. for 6 seconds and with the eater block which was set at 55° C. for 6 seconds. Amplification products were fractionated by electrophoresis on a 1.5% agarose gel. Total run time for the initial denaturation and 35 cycles of PCR was 10.7 minutes.

### Example 4

### Experiment 4

In experiment 4, the same reaction mixture was placed in the thermal cycling device and underwent an initial denaturation step where the PCR tube was contacted with the heater block which was set at 95° C. for 10 seconds. Then, the mixture was cycled 35 times through denaturation (95° C. temperature of first heater block) and annealing/extension (55° C. temperature of second heater block). During each cycle, the reaction tube was contacted with the which was set at 95° C. heater block for 6 seconds and with the which was set at 55° C. for 6 seconds. Amplification products were fractionated by electrophoresis on a 1.5% agarose gel. Total run time for the initial denaturation and 35 cycles of PCR was 10.7 minutes.

In experiment 1, electrophoresis showed that the expected 188-base-pair amplicon was produced. In experiment 2, no amplification product was observed with electrophoresis. In experiments 3 and 4, the expected 188-base-pair amplicon was observed with electrophoresis.

These results demonstrate that setting the temperature of the heater block at 99° C. (4° C. above the desired temperature of 95° C.) enabled the reaction to be run 12.6% faster than was achievable after thermal cycling with a 95° C. denaturation temperature of 95° C.

By setting the heater blocks at temperatures above and below the target temperatures for DNA denaturation and primer annealing/extension, it is possible to significantly reduce the amount of time for which the reaction vessel must be in contact with the heater block to reach the desired temperature.

This time savings enables PCR to be performed faster. For applications in which PCR results are time-sensitive, this may be particularly useful.

Also, the method of aspects of the present invention may be performed even when there is a large and non-uniform temperature gradient across the reaction vessel and reaction mixture because this enables the design of a mechanism in which heat is transferred to the reaction vessel with only partial heat-source contact. The requirement for partial rather than full contact enables the use of a mechanism that moves the heater blocks in one dimension, from side to side into grooves in heater blocks, rather than in two or three dimensions i.e. a robotic arm moving the reaction vessels up and down, and side to side, from one heater block to another.

Thus, it is seen that, contrary to conventional thinking, successful PRC was performed despite the presence of a very large non-uniform temperature gradient across the reaction vessel, i.e., 110° C. on one side of the reaction vessel and ambient air temperature on the other side.

It is also seen that, the reaction mixture in the liquid layer beside the reaction vessel surface in contact with the heater block which is set at 110° C. would have experienced local temperature conditions either reaching or very near to 110° C. It is known that temperatures in excess of 96° C. result in inactivation of DNA polymerase over time. Therefore, it might have been expected that more and more DNA polymerase in this surface liquid layer would have been inactivated with each PCR cycle, especially since thermal convection currents would have continually brought more active enzyme into contact with the inhospitably high 110° C. temperature. It would also be expected that this inactivation would prevent productive DNA amplification. However, neither of these expected inactivations occurred according to the method of aspects of the present invention.

### Conclusion

The foregoing has constituted a description of specific embodiments showing how the invention may be applied and put into use. These embodiments are only exemplary. The invention in its broadest, and more specific aspects, is further described and defined in the claims which follow.

These claims, and the language used therein are to be understood in terms of the variants of the invention which have been described. They are not to be restricted to such variants, but are to be read as covering the full scope of the invention as is implicit within the invention and the disclosure that has been provided herein.

1. A method of performing a nucleic acid amplification reaction where a reaction mixture is subjected sequentially to a selected denaturation temperature which is provided by a heat source and to a selected annealing, and/or extension temperature, said method comprising the step of moving said reaction mixture out of the influence of said heat source once said temperature is higher than said desired denaturation temperature and is lower than said desired annealing, and/or extension temperature.

2. The method according to claim 1, includes establishing a non-uniform temperature gradient across said reaction mixture.

3. The method according to claim 1, including the steps:
of first setting the temperature of a first heat source higher than the desired denaturation temperature, and setting the temperature of a second heat source lower than the desired annealing, and/or extension temperatures;
bringing the temperature of said reaction mixture to the desired denaturation temperature through the influence of said first heat source, and moving said reaction mixture out of the influence of said first heat source once the desired denaturation temperature is reached.

4. The method according to claim 2, including the steps:
of first setting the temperature of a first heat source higher than the desired denaturation temperature, and setting the temperature of a second heat source lower than the desired annealing, and/or extension temperatures;
bringing the temperature of said reaction mixture to the desired denaturation temperature through the influence of said first heat source, and moving said reaction mixture out of the influence of said first heat source once the desired denaturation temperature is reached.

5. The method according to claim 3, wherein the reaction mixture is brought to the desired denaturation temperature by direct contact with the heat source.

6. The method according to claim 4, wherein the reaction mixture is brought to the desired denaturation temperature by direct contact with the heat source.

7. The method according to claim 3, including the steps of:
setting the temperature of said first heat source to about 15° C. higher than said desired denaturation temperature; and subjecting said reaction mixture to the influence of said temperature; whereby the temperature of said reaction mixture is brought up to about 95° C. about 13 seconds.

8. The method according to claim 4, including the steps of:
setting the temperature of said first heat source to about 15° C. higher than said desired denaturation temperature; and subjecting said reaction mixture to the influence of said temperature; whereby the temperature of said reaction mixture is brought up to about 95° C. about 13 seconds.

9. The method according to claim 5, including the steps of:
setting the temperature of said first heat source to about 15° C. higher than said desired denaturation temperature; and subjecting said reaction mixture to the influence of said temperature; whereby the temperature of said reaction mixture is brought up to about 95° C. about 13 seconds.

10. The method according to claim 6, including the steps of:
setting the temperature of said first heat source to about 15° C. higher than said desired denaturation temperature; and subjecting said reaction mixture to the influence of said temperature; whereby the temperature of said reaction mixture is brought up to about 95° C. about 13 seconds.

11. The method according to claim 7, wherein the temperature of said reaction mixture is caused to increase by about 23° C. (65° C. to 88° C.) in only 13 seconds, i.e., at about 1.8 C/second.

12. The method according to claim 8, wherein the temperature of said reaction mixture is caused to increase by about 23° C. (65° C. to 88° C.) in only 13 seconds, i.e., at about 1.8 C/second.

13. The method according to claim 9, wherein the temperature of said reaction mixture is caused to increase by about 23° C. (65° C. to 88° C.) in only 13 seconds, i.e., at about 1.8 C/second.

14. The method according to claim 10, wherein the temperature of said reaction mixture is caused to increase by about 23° C. (65° C. to 88° C.) in only 13 seconds, i.e., at about 1.8 C/second.

15. The method according to claim 1, including:
setting the temperature of a second heat source to about 10° C. lower than the desired annealing, and/or extension temperatures; and subjecting the reaction mixture to the influence of said temperature for about 18 seconds, whereby the temperature of said reaction mixture is brought down to about 65° C.

16. The method according to claim 2, including:
setting the temperature of a second heat source to about 10° C. lower than the desired annealing, and/or extension temperatures; and subjecting the reaction mixture to the influence of said temperature for about 18 seconds, whereby the temperature of said reaction mixture is brought down to about 65° C.

17. The method according claim 3, including:
setting the temperature of a second heat source to about 10° C. lower than the desired annealing, and/or extension temperatures; and subjecting the reaction mixture to the influence of said temperature for about 18 seconds, whereby the temperature of said reaction mixture is brought down to about 65° C.

18. The method according to claim 4 including:
setting the temperature of a second heat source to about 10° C. lower than the desired annealing, and/or extension temperatures; and subjecting the reaction mixture to the influence of said temperature for about 18 seconds, whereby the temperature of said reaction mixture is brought down to about 65° C.

19. The method according to claim 5, including:
setting the temperature of a second heat source to about 10° C. lower than the desired annealing, and/or extension temperatures; and subjecting the reaction mixture to the influence of said temperature for about 18 seconds, whereby the temperature of said reaction mixture is brought down to about 65° C.

20. The method according to claim 6, including:
setting the temperature of a second heat source to about 10° C. lower than the desired annealing, and/or extension temperatures; and subjecting the reaction mixture to the influence of said temperature for about 18 seconds, whereby the temperature of said reaction mixture is brought down to about 65° C.

21. The method according to claim 7, including:
setting the temperature of a second heat source to about 10° C. lower than the desired annealing, and/or extension temperatures; and subjecting the reaction mixture to the influence of said temperature for about 18 seconds, whereby the temperature of said reaction mixture is brought down to about 65° C.

22. The method according to claim 8, including:
setting the temperature of a second heat source to about 10° C. lower than the desired annealing, and/or extension temperatures; and subjecting the reaction mixture to the influence of said temperature for about 18 seconds, whereby the temperature of said reaction mixture is brought down to about 65° C.

23. The method according to claim 9, including:
setting the temperature of a second heat source to about 10° C. lower than the desired annealing, and/or extension temperatures; and subjecting the reaction mixture to the influence of said temperature for about 18 seconds, whereby the temperature of said reaction mixture is brought down to about 65° C.

24. The method according to claim 10, including:
setting the temperature of a second heat source to about 10° C. lower than the desired annealing, and/or extension temperatures; and subjecting the reaction mixture to the influence of said temperature for about 18 seconds, whereby the temperature of said reaction mixture is brought down to about 65° C.

25. The method according to claim 11, including:
setting the temperature of a second heat source to about 10° C. lower than the desired annealing, and/or extension temperatures; and subjecting the reaction mixture to the influence of said temperature for about 18 seconds, whereby the temperature of said reaction mixture is brought down to about 65° C.

26. The method according to claim 12, including:
setting the temperature of a second heat source to about 10° C. lower than the desired annealing, and/or extension temperatures; and subjecting the reaction mixture to the influence of said temperature for about 18 seconds, whereby the temperature of said reaction mixture is brought down to about 65° C.

27. The method according to claim 13, including:
setting the temperature of a second heat source to about 10° C. lower than the desired annealing, and/or extension temperatures; and subjecting the reaction mixture to the influence of said temperature for about 18 seconds, whereby the temperature of said reaction mixture is brought down to about 65° C.

28. The method according to claim 14 including: setting the temperature of a second heat source to about 10° C. lower than the desired annealing, and/or extension temperatures; and subjecting the reaction mixture to the influence of said temperature for about 18 seconds, whereby the temperature of said reaction mixture is brought down to about 65° C.
